(19) United States
(12) Patent Application Publication
(10) Pub. No.: US 2019/0134034 A1
(43) Pub. Date: May 9, 2019

(54) Method of Treating Liver Cancer

(71) Applicant: ASLAN Pharmaceuticals Pte. Ltd., Singapore (SG)

(72) Inventors: Lisa Ooi, Singapore (SG); Bertil Lindmark, Singapore (SG)

(73) Assignee: ASLAN Pharmaceuticals Pte. Ltd., Singapore (SG)

(21) Appl. No.: 16/094,199

(22) PCT Filed: Apr. 21, 2017
(86) PCT No.: PCT/SG2017/050227

§ 371 Date: Oct. 16, 2018

(30) Foreign Application Priority Data
Apr. 21, 2016 (GB) 1606984.1
May 17, 2016 (GB) 1608658.9
Jul. 1, 2016 (GB) 1611579.2

(51) Int. Cl. A61K 31/513; A61P 35/00; A61K 31/455; A61K 31/4745; A61K 31/517; A61K 31/519; A61K 31/555; A61K 31/704; A61K 31/7068; A61K 33/24

(52) U.S. Cl. CPC A61K 31/513; A61P 35/00; A61K 31/455; A61K 31/4745; A61K 33/24; A61K 31/519; A61K 31/555; A61K 31/704; A61K 31/7068; A61K 31/517

(57) ABSTRACT

A method of treating liver cancer, such as hepatocellular carcinoma, by administering a compound of formula (I), such as Varlitinib, or an enantiomer thereof, or a pharmaceutically acceptable salt thereof. Also provided is a compound of formula (I) for use in the treatment of liver cancer and use of a compound of formula (I) in the manufacture of a medicament for the treatment of liver cancer.

The present disclosure relates to a therapy, in particular a monotherapy comprising a type I tyrosine kinase inhibitor for the treatment of liver cancer, such as hepatocellular carcinoma (HCC), and variants thereof.

### BACKGROUND

Liver cancer, in particular hepatocellular carcinoma (HCC) is the fifth and eighth most common malignancy in men and women respectively in the world. Liver cancer accounts for 662,000 deaths each year, which represents about a third of the cancer-related deaths. It is more common than breast cancer and colon cancer. Approximately 75 to 80% of cases of HCC occur in Asia.

The American Cancer Society's estimates for primary liver cancer and intrahepatic bile duct cancer in the United States for 2015 are:
about 35,660 new cases (25,510 in men and 10,150 in women) will be diagnosed, and
about 24,550 people (17,030 men and 7,520 women) will die of these cancers.

The average age at diagnosis of liver cancer is 63. More than 95% of people diagnosed with liver cancer are 45 years of age or older. About 3% are between 35 and 44 years of age and about 2% are younger than 35.

The usual prognosis is poor because only 10-20% of hepatocellular carcinomas can be removed completely by surgery. If the cancer cannot be completely removed, the disease is usually deadly within 3 to 6 months. This is partially due to late diagnosis of patients with large tumours, but is also due to the lack of medical expertise and facilities in the regions with high HCC prevalence. However, survival can vary, and occasionally people will survive much longer than 6 months. The prognosis for metastatic or unresectable hepatocellular carcinoma has recently improved due to the approval of sorafenib (Nexavar®) for advanced hepatocellular carcinoma.

Hepatitis C is a significant risk factor for HCC. An estimated 150-200 million people worldwide are infected with hepatitis C and about 343,000 deaths each year are due to liver cancer from hepatitis C. There is no effective vaccine against hepatitis C available.

Other etiologies include hepatitis B, alcoholic cirrhosis, haemochromatosis, autoimmune hepatitis, biliary cirrhosis, and aflatoxin B ingestion. Due to the geo-cultural and varying patterns of liver infection, genetic background and food intake there are major geographical differences in the incidence of HCC across the globe.

Accordingly, there is a need for new chemotherapeutic agents and therapies for treating liver cancer, such as hepatocellular carcinoma.

### SUMMARY OF THE DISCLOSURE

The present disclosure provides a method of treating a liver cancer patient, for example a hepatocellular carcinoma patient by administering a therapeutically effective amount of a compound of formula (I):

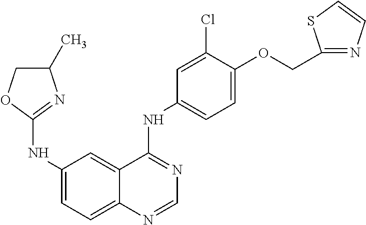 [SMILES: CC1COC(Nc2ccc3ncnc(Nc4ccc(OCc5nccs5)c(Cl)c4)c3c2)=N1]

an enantiomer thereof and pharmaceutically acceptable salts thereof. The present inventors have data that suggests there is dysregulation of HER signaling in HCC and that a pan-HER inhibitor, such as compound of formula (I), would be useful in the treatment of the same.

In one embodiment the compound of formula (I) is (R)-N4-[3-Chloro-4-(thiazol-2-ylmethoxy)-phenyl]-N6-(4-methyl-4,5,-dihydro-oxazol-2-yl)-quinazoline-4,6-diamine also known as Varlitinib:

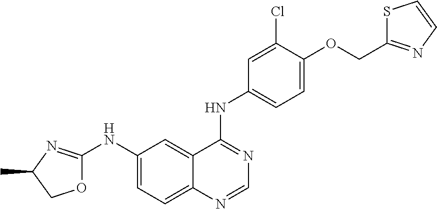 [SMILES: C[C@@H]1COC(Nc2ccc3ncnc(Nc4ccc(OCc5nccs5)c(Cl)c4)c3c2)=N1]

or a pharmaceutically acceptable salt thereof or a pro-drug thereof.

In one embodiment, the compound of formula (I) is provided as the free base.

In one embodiment, the compound of formula (I) is administered as a pharmaceutical formulation.

In one embodiment, the compound of formula (I) or a pharmaceutical formulation comprising the same is administered bi-daily.

In one embodiment the compound of formula (I) is administered bi-daily, for example at a dose in the range 100 mg to 900 mg on each occasion, in particular 100 mg, 200 mg, 300 mg, 400 mg or 500 mg each dose.

In one embodiment the compound of formula (I) is administered once daily, for example at a dose in the range 100 mg to 900 mg on each occasion, in particular 100 mg, 200 mg, 300 mg, 400 mg or 500 mg each dose.

In one embodiment the compound of formula (I) is administered once a week, for example at a dose disclosed herein.

In one embodiment the doses of the compound of formula (I) is administered bi-daily, for example 2-6 days per week, for example 2, 3, 4, 5 or 6 days per week, in particular at a dose in the range 100 mg to 900 mg on each occasion, in particular 100 mg, 200 mg, 300 mg, 400 mg or 500 mg each dose.

In one embodiment the compound of formula (I) is administered once daily, for 2-6 days per week, for example 2, 3, 4, 5 or 6 days per week, in particular at a dose in the range 100 mg to 900 mg on each occasion, in particular 100 mg, 200 mg, 300 mg, 400 mg or 500 mg each dose.

In one embodiment the dose of a compound of formula (I) is 25 to 100 mg/Kg, for example 30, 35, 40, 45, 50, 55, 60, 65, 70, 75, 80, 85, 90, 95 or 100 mg/Kg.

In one embodiment the compound of formula (I) is administered as pharmaceutical formulation comprising one or more pharmaceutically acceptable excipients.

In one embodiment the compound of formula (I) or formulation comprising the same is administered orally, for example as a tablet or a capsule.

In one embodiment the compound of formula (I) is employed as a monotherapy.

In one embodiment the compound of formula (I) is employed as part of a combination therapy.

In one embodiment the combination therapy comprises a chemotherapeutic agent, for example selected from the group comprising doxorubicin, a platin (such as cisplatin or oxaliplatin), gemcitabine, capecitabine, 5-FU, FOLFOX, FOLFIRI and FOLFIRINOX.

In one embodiment, the combination therapy comprises a targeted therapy selected from the group comprising sorafenib or a FGFR inhibitor.

In one embodiment the combination therapy comprises a PARP inhibitor.

In one embodiment the target patient population is EGFR and HER2 positive or are HER2 amplified.

In one embodiment the patient population is HER 1 positive.

In one embodiment the patient population is HER 2 positive.

In one embodiment the patient population is HER 3 positive.

In one embodiment the patient population is HER 4 positive.

In one embodiment said cancer cells have increased levels of HER2 phosphorylation.

In one embodiment said cancer cells have increased levels of HER1 phosphorylation.

In one embodiment said cancer cells have increased levels of HER3 phosphorylation.

In one embodiment said cancer have increased levels of HER4 phosphorylation.

In one embodiment, the patient population for treatment has HER pathway activation indicated by high levels of phosphorylated downstream signalling proteins, for example selected from pAKT and pERK.

In one embodiment the treatment is adjuvant therapy, for example after surgery or after chemotherapy.

In one embodiment the treatment neo-adjuvant therapy.

In one embodiment the therapy according to the present disclosure is employed in a combination therapy.

In one embodiment the therapy according to the present disclosure is not administered concomitantly with chemotherapy.

In one embodiment the therapy according to the present disclosure is not administered as a chemosensitizing agent

In one embodiment the therapy according to the present disclosure is employed in combination with a DHODH inhibitor.

In one embodiment, the DHODH inhibitor is selected from the group comprising teriflunomide, leflunomide a compound of formula (II) (disclosed in WO2008/077639 incorporated herein by reference):

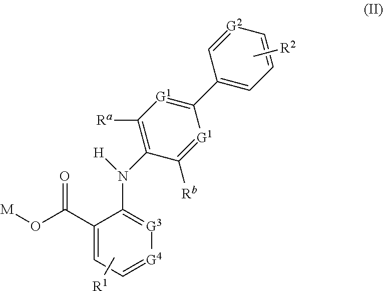

wherein:
one of the groups G¹ represents a nitrogen atom or a group CR^{c} and the other group represents CR^{c};
G² represents a nitrogen atom or a group CR^{d};
R¹ represents a group selected from hydrogen, halogen, C₁₋₄ alkyl which may be optionally substituted with 1, 2 or 3 substituents selected from the group comprising halogen, hydroxy, and C₃₋₈ cycloalkyl which may be optionally substituted with 1, 2 or 3 substituents selected from halogen and hydroxyl;
R² represents a group selected from hydrogen, halogen, hydroxyl, C₁₋₄ alkyl which may be optionally substituted by 1, 2 or 3 substituents selected from the group comprising halogen, hydroxy, C₃₋₈ alkyl which may be optionally substituted with 1, 2, or 3 substituents selected from halogen and hydroxyl;
R^{a}, R^{b} and R^{c} independently represent a radical selected from the group comprising hydrogen, halogen, C₁₋₄ alkyl optionally substituted by 1, 2 or 3 substituents selected from the group comprising halogen, hydroxy and C₁₋₄ alkoxy;
R^{d} represents a group selected from hydrogen, halogen, hydroxyl, C₁₋₄ alkyl which may be substituted by 1, 2 or 3 substituents selected from the group comprising halogen, hydroxyl, C₁₋₄ alkoxy which may be optionally substituted with 1, 2 or 3 substituent selected from the group comprising halogen, hydroxy, and C₃₋₈ cycloalkoxy which may be optionally substituted with 1, 2 or 3 substitutents selected from halogen and hydroxyl;
G³ & G⁴ one is a nitrogen atom and the other is a CH;
M is hydrogen or a pharmaceutically acceptable cation.
In one embodiment the compound of formula (II) has the proviso that, when at least one of the groups R^{a} and R^{b} represents a hydrogen atom and G² is a group CR^{d}, then R^{d} represents a group selected from C₁₋₄ alkoxy which may be optionally substituted with 1, 2 or 3 substituents selected from halogen, hydroxy, C₃₋₈ cycloalkoxy which may be optionally substituted with 1, 2 or 3 substituents selected from halogen and hydroxyl.
In one embodiment the DHODH inhibitor is 2-(3, 5-difluoro-3′-methoxybiphenyl-4-ylamino) nicotinic acid (referred to herein as ASLAN003) or a pharmaceutically acceptable salt thereof, in particular:

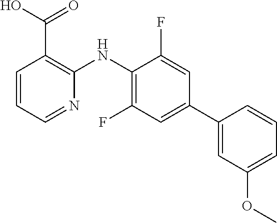 [SMILES: COc1cccc(-c2cc(F)c(Nc3ncccc3C(=O)O)c(F)c2)c1]

In one embodiment the therapy according to the present disclosure is not employed in combination with a DHODH inhibitor.

In one embodiment the tumour is a solid tumour. In one embodiment the treatment according to the present disclosure is suitable for the treatment of secondary tumours. In one embodiment the cancer is metastatic cancer. In one embodiment the treatment according to the present disclosure is suitable for the treatment of primary cancer and metastases.

In one embodiment the therapy of the present disclosure is administered to treat primary liver cancer.

In one embodiment the therapy of the present disclosure is administered to treat secondary liver cancer.

In one embodiment the liver cancer is hepatocellular carcinoma.

In one embodiment the patient is a refractory cancer patient, for example a patient whose cancer exhibits resistance to other anti-cancer therapies, such a chemotherapy.

In one embodiment the patient is a mammal, for example a human.

In one embodiment the human patient is a an adult, for example over the age of 18. In one embodiment the human patient is a child or adolescent, for example under the age of 18.

In one aspect there is provided use of a compound of formula (I), an enantiomer thereof or a pharamaceutically acceptable salt thereof in the treatment of liver cancer, for example hepatocellular carcinoma, in particular as described herein.

In one aspect there is provided use of a compound of formula (I), an enantiomer thereof or a pharamaceutically acceptable salt thereof in the manufacture of a medicament for the treatment of liver cancer, for example hepatocellular carcinoma, in particular as described herein.

In one embodiment the therapy continues for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 months or more.

### BRIEF SUMMARY OF THE FIGURES

FIG. 1 shows the pharmacodynamic results from a HCC patient-derived xenograft model HCC29-0909A after 2 days of varlitinib monotherapy at different doses.

FIG. 2 shows the pharmacodynamic results from a HCC patient-derived xenograft model HCC29-0909A after day 14 of varlitininb monotherapy at different doses.

FIG. 3 shows dose-dependent tumour volume growth inhibition in HCC patient derived xenograft model of HCC29-0909A after administration of 25 mg/kg BID, 50 mg/kg BID or 100 mg/kg BID of varlitinib.

FIG. 4 shows the pharmacodynamic results from a HCC patient-derived xenograft model HCC01-0708 after 2 days of varlitinib monotherapy.

FIG. 5 shows the pharmacodynamic results from a HCC patient-derived xenograft model HCC01-0708 after day 12 of varlitinib monotherapy at different doses.

FIG. 6 shows dose-dependent tumour volume growth inhibition in HCC patient derived xenograft model of HCC01-0708 after administration of 25 mg/kg BID, 50 mg/kg BID or 100 mg/kg BID of varlitinib.

FIG. 7 shows the results of an in vitro experiment to investigate the induction of apoptosis by varlitinib in HCC cell lines (including sorafenib-resistant cell lines.

FIG. 8 shows the apoptosis (AnnexinV) profile for PLC/PRF/5 cells after 48 hour culture in the presence of varlitinib.

### DETAILED DESCRIPTION OF THE DISCLOSURE

Embodiments disclosed herein related to compounds of formula (I) also extend explicitly to varlitinib.

An enantiomer as employed herein refers to where one enantiomer, for example the R enantiomer or the S enantiomer, in particular the R enantiomer is provided in enantiomeric excess, for example more than 50%, enantiomeric excess, such as 60, 65, 70, 75, 80, 85, 90, 95, 96, 97, 98, 99% enantiomeric excess.

The compound of formula (I) disclosed herein is a pan-HER inhibitor.

Pan-HER inhibitor as employed herein refers to a molecule that inhibits at least two molecules from the ErbB family of proteins, namely ErbB-1 (also known as HER1 and EGFR), ErbB-2 (HER2), ErbB-3 (HER3), and ErbB-4(HER4).

In one embodiment the compound of formula (I) at least inhibits the activity of HER1 and HER2, HER1 and HER4 or HER2 and HER4.

In one embodiment the compound of formula (I) at least inhibits the activity of HER1 and HER3, HER2 and HER3 or HER3 and HER4.

In one embodiment the compound of formula (I) at least inhibits the activity of HER1, HER2, and HER3.

In one embodiment the compound of formula (I) at least inhibits the activity of HER1, HER2 and HER4, for example directly inhibits the activity of HER1, HER2 and HER4.

In one embodiment the compound of formula (I) inhibits the activity of HER1, HER2, HER3 and HER4, for example directly inhibits the activity of HER1, HER2, and HER4, and indirectly inhibits the activity of HER3.

Inhibitor as employed refers to the reduction of a relevant biological activity, for example by 5, 10, 15, 20, 25, 30, 35, 40, 45, 50, 55, 60, 65, 70, 75, 80, 85, 90, 95 or 100%, such as when measured in a relevant in vitro assay.

Direct inhibition is where the inhibitor binds directly to or physically blocks a binding interaction to inhibit a biological activity, or when the inhibitor inhibits the activation through phosphorylation of the target molecule.

Indirect inhibition as employed herein refers to where the biological activity in question is inhibited as a result of directly inhibiting a target that is other than the entity that is indirectly inhibited.

Liver cancer as used herein Liver cancer as employed herein refers to cancer of the liver, for example hepatocellular carcinoma, fibrolamellar carcinoma, cholangiocarcinoma, angiosarcoma and hepatoblastoma.

In one embodiment the liver cancer is hepatocellular carcinoma (HCC).

In one embodiment the cancer is fibrolamellar carcinoma.

Primary liver cancer as employed herein is a cancer that starts or originates in the liver.

In one embodiment the cancer does not extend to cholangiocarcinoma.

In one embodiment the cancer is bile duct cancer.

Secondary liver cancer as employed herein is a cancer that starts or originate outside the liver and spreads to the liver.

Treatment as employed herein refers to where the patient has a disease or disorder, for example cancer and the medicament according to the present disclosure is administered to stabilise the disease, delay the disease, ameliorate the disease, send the disease into remission, maintain the disease in remission or cure the disease.

Prophylaxis as employed herein refers to administering the medicament according to the present disclosure to prevent the development of a disease, such as cancer. Treating as employed herein includes administration of a medicament according to the present disclosure for treatment or prophylaxis.

A therapeutically effective amount as employed herein refers to a dose in the context of treatment or prophylaxis which elicits the desired pharmacological effect.

Sensitizing a cancer patient to chemotherapy as employed herein refers to increasing the patient's response to chemotherapy, or where the patient is resistant to chemotherapy rendering the cancer susceptible to chemotherapy.

Combination therapy as employed herein refers to where a medicament according to the present disclosure is administered in a treatment regimen along with at least one further therapeutic agent. The regime may be separate formulations administered at the same time or different times or co-formulations of the two or more therapeutic agents. The medicament according to the present disclosure may be administered; prior to the further therapeutic agent or agents, concomitant with the further therapeutic agent or agents, or after the further therapeutic agent or agents.

In one embodiment the further therapeutic agent or agents is/are an anti-cancer therapy.

Not employed concomitantly with a chemotherapeutic agent as employed herein refers to the fact that the treatment regimen of the present disclosure does not overlap with a treatment regimen for a chemotherapeutic agent.

Chemotherapeutic agent and chemotherapy or cytotoxic agent are employed interchangeably herein unless the context indicates otherwise.

In one embodiment further therapeutic agent or agents, such as an anti-cancer therapy are employed in combination with the therapy of the present disclosure.

Chemotherapeutic agent as employed herein is intended to refer to specific antineoplastic chemical agents or drugs that are destructive to malignant cells and tissues, including alkylating agents, antimetabolites, anthracyclines, plant alkaloids, topoisomerase inhibitors, and other antitumour agents. Specific examples of chemotherapy include sorafenib, doxorubicin, 5-fluorouracil (5-FU), paclitaxel (for example abraxane or docetaxel), capecitabine, irinotecan, and platins, such as cisplatin and oxaliplatin or a combination thereof.

The preferred dose may be chosen by the practitioner, based on the nature of the cancer being treated.

Examples of alkylating agents, which may be employed in the method of the present disclosure include an alkylating agent nitrogen mustards, nitrosoureas, tetrazines, aziridines, platins and derivatives, and non-classical alkylating agents.

Examples of a platinum containing chemotherapeutic agent (also referred to as platins), such as cisplatin, carboplatin, oxaliplatin, satraplatin, picoplatin, nedaplatin, triplatin and lipoplatin (a liposomal version of cisplatin), in particular cisplatin, carboplatin and oxaliplatin.

The dose for cisplatin ranges from about 20 to about 270 mg/m² depending on the exact cancer. Often the dose is in the range about 70 to about 100 mg/m².

Nitrogen mustards include mechlorethamine, cyclophosphamide, melphalan, chlorambucil, ifosfamide and busulfan.

Nitrosoureas include N-Nitroso-N-methylurea (MNU), carmustine (BCNU), lomustine (CCNU) and semustine (MeCCNU), fotemustine and streptozotocin. Tetrazines include dacarbazine, mitozolomide and temozolomide.

Aziridines include thiotepa, mytomycin and diaziquone (AZQ).

Examples of antimetabolites, which may be employed in the method of the present disclosure, include anti-folates (for example methotrexate and pemetrexed), purine analogues (for example thiopurines, such as azathiopurine, mercaptopurine, thiopurine, fludarabine (including the phosphate form), pentostatin and cladribine), pyrimidine analogues (for example fluoropyrimidines, such as 5-fluorouracil and prodrugs thereof such as capecitabine [Xeloda®]), floxuridine, gemcitabine, cytarabine, decitabine, raltitrexed(tomudex) hydrochloride, cladribine and 6-azauracil.

Examples of anthracyclines, which may be employed in the method of the present disclosure, include daunorubicin (Daunomycin), daunorubicin (liposomal), doxorubicin (Adriamycin), doxorubicin (liposomal), epirubicin, idarubicin, valrubicin currenity used only to treat bladder cancer and mitoxantrone an anthracycline analog, in particular doxorubicin.

Examples of anti-microtubule agents, which may be employed in the method of the present disclosure, include include vinca alkaloids and taxanes.

Vinca alkaloids include completely natural chemicals for example vincristine and vinblastine and also semi-synthetic vinca alkaloids, for example vinorelbine, vindesine, and vinflunine

Taxanes include paclitaxel, docetaxel, abraxane, carbazitaxel and derivatives of thereof. Derivatives of taxanes as employed herein includes reformulations of taxanes like taxol, for example in a micelluar formulaitons, derivatives also include chemical derivatives wherein synthetic chemistry is employed to modify a starting material which is a taxane.

Topoisomerase inhibitors, which may be employed in a method of the present disclosure include type I topoisomerase inhibitors, type II topoisomerase inhibitors and type II topoisomerase poisons. Type I inhibitors include topotecan, irinotecan, indotecan and indimitecan. Type II inhibitors include genistein and ICRF 193 which has the following structure:

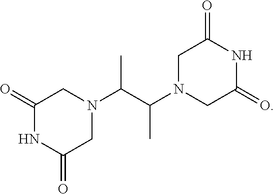 [SMILES: CC(C(C)N1CC(=O)NC(=O)C1)N1CC(=O)NC(=O)C1]

Type II poisons include amsacrine, etoposide, etoposide phosphate, teniposide and doxorubicin and fluoroquinolones.

In one embodiment the chemotherapeutic is a PARP inhibitor.

In one embodiment a combination of chemotherapeutic agents employed is, for example a platin and 5-FU or a prodrug thereof, for example cisplatin or oxaplatin and capecitabine or gemcitabine, such as FOLFOX.

In one embodiment the chemotherapy comprises a combination of chemotherapy agents, in particular cytotoxic chemotherapeutic agents.

In one embodiment the chemotherapy combination comprises a platin, such as cisplatin and fluorouracil or capecitabine.

In one embodiment the chemotherapy combination in capecitabine and oxaliplatin (Xelox).

In one embodiment the chemotherapy is a combination of folinic acid and 5-FU, optionally in combination with oxaliplatin.

In one embodiment the chemotherapy is a combination of folinic acid, 5-FU and irinotecan (FOLFIRI), optionally in combination with oxaliplatin (FOLFIRINOX). The regimen consists of: irinotecan (180 mg/m² IV over 90 minutes) concurrently with folinic acid (400 mg/m² [or 2×250 mg/m²] IV over 120 minutes); followed by fluorouracil (400-500 mg/m² IV bolus) then fluorouracil (2400-3000 mg/m² intravenous infusion over 46 hours). This cycle is typically repeated every two weeks. The dosages shown above may vary from cycle to cycle.

In one embodiment the chemotherapy combination employs a microtubule inhibitor, for example vincristine sulphate, epothilone A, N-[2-[(4-Hydroxyphenyl)amino]-3-pyridinyl]-4-methoxybenzenesulfonamide (ABT-751), a taxol derived chemotherapeutic agent, for example paclitaxel, abraxane, or docetaxel or a combination thereof.

In one embodiment the chemotherapy combination employs an mTor inhibitor. Examples of mTor inhibitors include: everolimus (RAD001), WYE-354, KU-0063794, papamycin (Sirolimus), Temsirolimus, Deforolimus(MK-8669), AZD8055 and BEZ235(NVP-BEZ235).

In one embodiment the chemotherapy combination employs a MEK inhibitor. Examples of MEK inhibitors include: AS703026, CI-1040 (PD184352), AZD6244 (Selumetinib), PD318088, PD0325901, AZD8330, PD98059, U0126-EtOH, BIX 02189 or BIX 02188. In one embodiment the chemotherapy combination employs an AKT inhibitor. Examples of AKT inhibitors include: MK-2206 and AT7867.

In one embodiment the combination employs an aurora kinase inhibitor. Examples of aurora kinase inhibitors include: Aurora A Inhibitor I, VX-680, AZD1152-HQPA (Barasertib), SNS-314 Mesylate, PHA-680632, ZM-447439, CCT129202 and Hesperadin.

In one embodiment the chemotherapy combination employs a p38 inhibitor, for example as disclosed in WO2010/038086, such as N-[4-({4-[3-(3-tert-Butyl-1-p-tolyl-1H-pyrazol-5-yl)ureido]naphthalen-1-yloxy}methyl)pyridin-2-yl]-2-methoxyacetamide.

In one embodiment the combination employs a Bcl-2 inhibitor. Examples of Bcl-2 inhibitors include: obatoclax mesylate, ABT-737, ABT-263(navitoclax) and TW-37.

In one embodiment the chemotherapy combination comprises an antimetabolite such as capecitabine (xeloda), fludarabine phosphate, fludarabine (fludara), decitabine, raltitrexed (tomudex), gemcitabine hydrochloride and cladribine.

In one embodiment the chemotherapy combination comprises ganciclovir, which may assist in controlling immune responses and/or tumour vasculation.

In one embodiment the chemotherapy includes a PARP inhibitor.

In one embodiment one or more therapies employed in the method herein are metronomic, that is a continuous or frequent treatment with low doses of anticancer drugs, often given concomitant with other methods of therapy.

In one embodiment, there is provided the use of multiple cycles of treatment (such as chemotherapy) for example 2, 3, 4, 5, 6, 7, 8.

In one embodiment the combination of the present disclosure is employed after chemotherapy.

In one embodiment the combination therapy of the present disclosure is employed before chemotherapy.

In one embodiment the dose of chemotherapy employed in the combination therapy of the present disclosure is lower than the dose of chemotherapy employed in “monotherapy” (where monotherapy may include the dose of chemotherapy employed when combinations of chemotherapy agents are employed).

In one embodiment the therapy according to the present disclosure is administered in combination with therapy complimentary to the cancer therapy, for example a treatment for cachexia, such as cancer cachexia, for example S-pindolol, S-mepindolol or S-bopindolol. Suitable doses may be in the range of 2.5 mg to 100 mg, such as 2.5 mg to 50 mg per day provided a single dose or multiple doses given as multiple doses administered during the day.

In one embodiment the therapy according to the present disclosure is administered in combination with a PD-1 or a PDL-1, or any other immune checkpoint inhibitor.

In one embodiment the therapy according to the present disclosure is employed in combination with surgery to remove part or all of the cancer tissue, for example the treatment of the present disclosure is adjuvant therapy following surgery or neo-adjuvant therapy, for example to increase the likely success of the surgery. The latter may be achieved by, for example shrinking a tumor or reducing the likelihood of metastasis or similar.

A DHODH inhibitor as employed herein refers to a compound which inhibits the activity of dihydroorotate dehydrogenase, in particular in vivo. Compounds of formula (II) described above are examples of DHODH inhibitors. These compounds are disclosed in WO2008/077639, incorporated herein by reference.

Other examples of DHODH inhibitor, which may be employed in a methods of the present disclosure include:
teriflunomide which has the following structure:

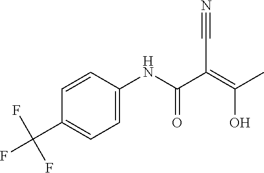 [SMILES: C/C(O)=C(\C#N)C(=O)Nc1ccc(C(F)(F)F)cc1]

and the compounds disclosed in WO97/34600 incorporated herein by reference;
leflunomide which has the following structure:

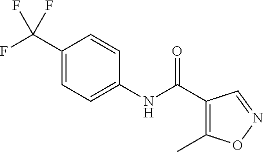 [SMILES: Cc1oncc1C(=O)Nc1ccc(C(F)(F)F)cc1]

the DHODH inhibitors of formula (1) disclosed in WO99/45926 incorporated herein by reference;
compounds of formula (I) disclosed in WO2003/006425 incorporated herein by reference;
the DHODH inhibitors of formula (I) disclosed in WO2004/056746 incorporated herein by reference;
compounds of formula (I) disclosed in WO2006/022442 incorporated herein by reference; and
DHODH inhibitors disclosed in WO2009/021696 incorporated herein by reference.

Suitable salts of DHODH inhibitors include those disclosed in WO2010/102826, WO2010/10225 and WO2010/102824 each incorporated herein by reference.

In the context of this specification “comprising” is to be interpreted as “including”.

Aspects of the disclosure comprising certain elements are also intended to extend to alternative embodiments “consisting” or “consisting essentially” of the relevant elements.

Positively recited embodiments may be employed herein as a basis for a disclaimer.

All references referred to herein are specifically incorporated by reference.

The invention will now be described with reference to the following examples, which are merely illustrative and should not in any way be construed as limiting the scope of the present disclosure.

### EXAMPLES

### Example 1

A HCC patient-derived xenograft model in SCID mice (HCC29-0909A) with co-expression of HER1, HER2 and HER3 receptors [Cancer Res Aug. 1, 2015; 752674 (AACR Abstract 2674): Activity of BAY1082439, a balanced PI3Ka/b inhibitor, in gastric cancer Huynh T. Hung, Richard Ong, Katja Haike, Elissaveta Petrova, Mei Ling Chong Marie Loh, Bhaskar Bhattacharya, Richie Soong and Ningshu Liu.] was used to evaluate the pan-HER inhibitor varlitinib, which has the chemical name (R)-N4-[3-Chloro-4-(thiazol-2-ylmethoxy)-phenyl]-N6-(4-methyl-4,5,-dihydro-oxazol-2-yl)-quinazoline-4,6-diamine (ASLAN001). Dosing with varlitinib commenced when the tumours reached the size of approximately 100-150 mm³. Bi-dimensional measurements are performed twice a week and tumour volumes are calculated based on the following formula: Tumour volume=[(Length)×(Width²)×(p/6)].

Groups of mice were dosed as follows:

Group 1 control group received vehicle only

Group 2 received 25 mg/Kg of ASLAN001 BID (twice daily),

Group 3 received 50 mg/Kg of ASLAN001 BID (twice daily), and

Group 4 received 100 mg/Kg of ASLAN001 BID (twice daily).

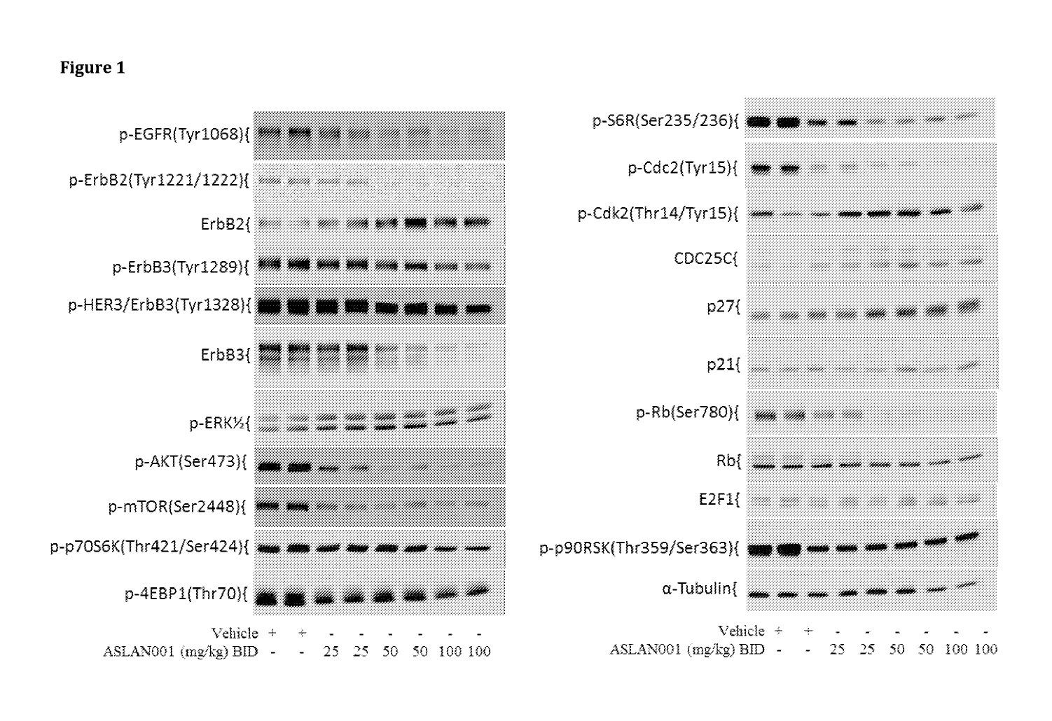

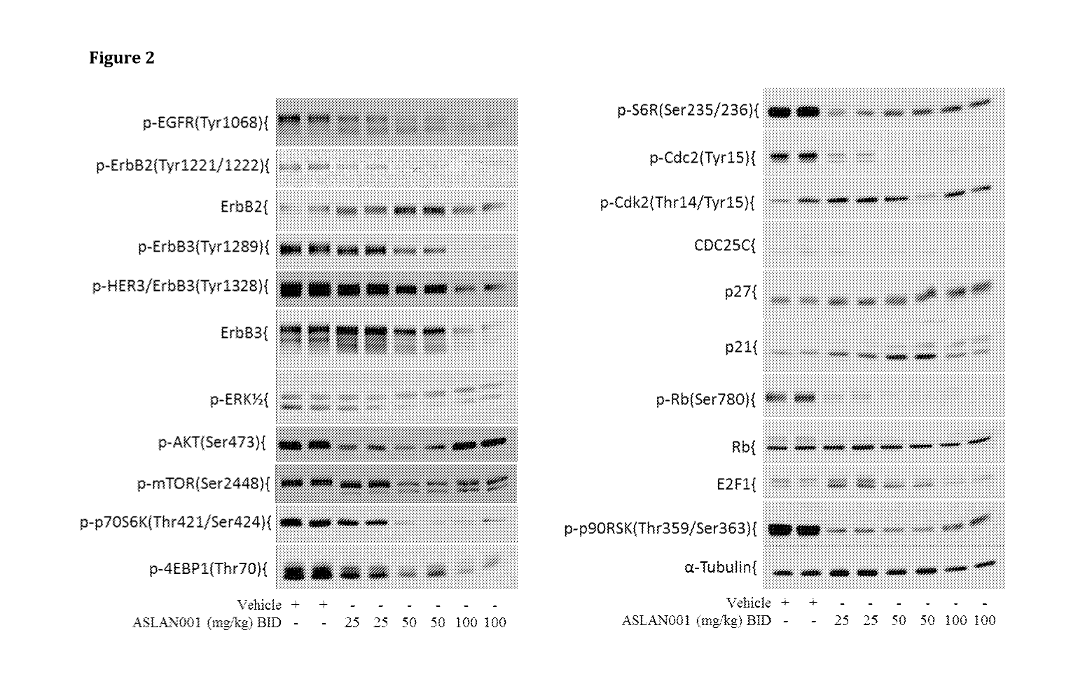

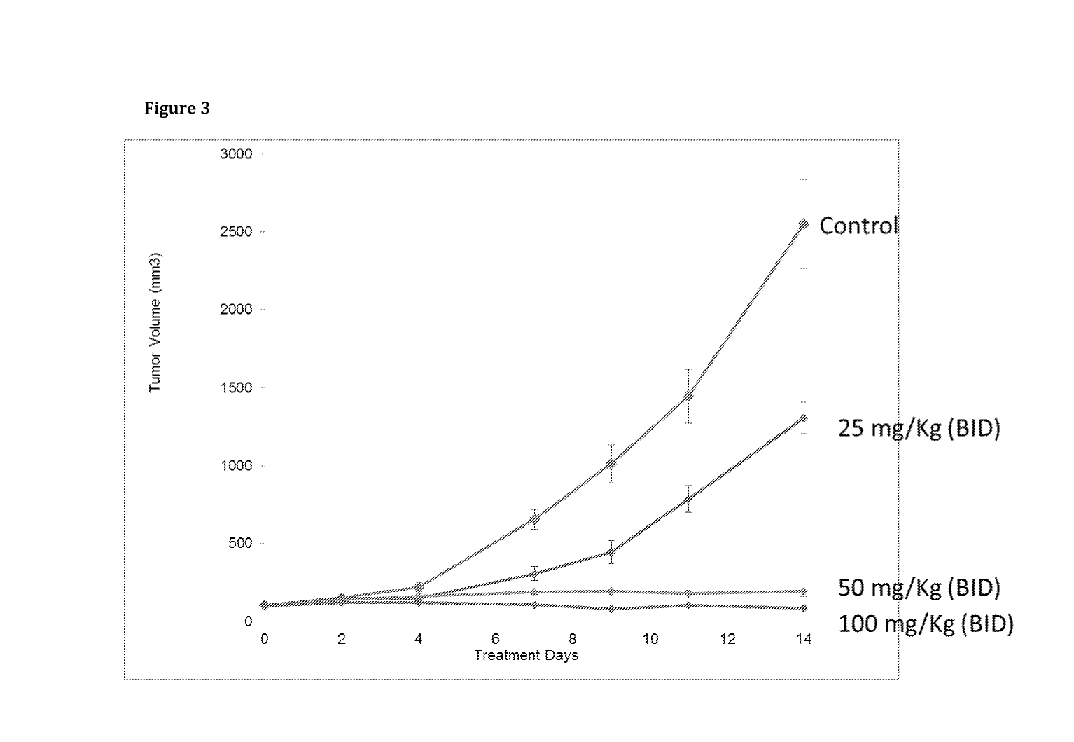

The results are shown in FIGS. 1 to 3.

Results:

Varlitinib treatment potently inhibited tumour growth with complete tumor regression observed at dosing of 100 mg/kg BID. In addition, varlitinib was well tolerated at all dose levels. Western blot analysis of tumour lysates taken after two and fourteen days of varlitinib treatment revealed that phosphorylation of HER1-3, RAS/RAF/MEK/MAPK, p70S6K, S6 ribosomal, 4EBP1, Cdc-2 and retinoblastoma were strongly inhibited.

Conclusions:

Our data suggest that varlitinib potently inhibited cancer cell proliferation and modulated several growth and survival pathways in the liver cancer PDX model. In the clinical setting varlitinib has already demonstrated impressive efficacy in breast cancer, gastric cancer, cholangiocarcinoma and colorectal carcinoma with a greatly improved toxicity profile compared to irreversible pan-HER inhibitors such as Neratinib and Dacomitinib. Given the robust and tolerable anti-tumour activity of varlitinib in PDX models of HCC, this indicates that varlitinib has the potential to be useful for the treatment of liver cancer in the clinic.

### Example 2

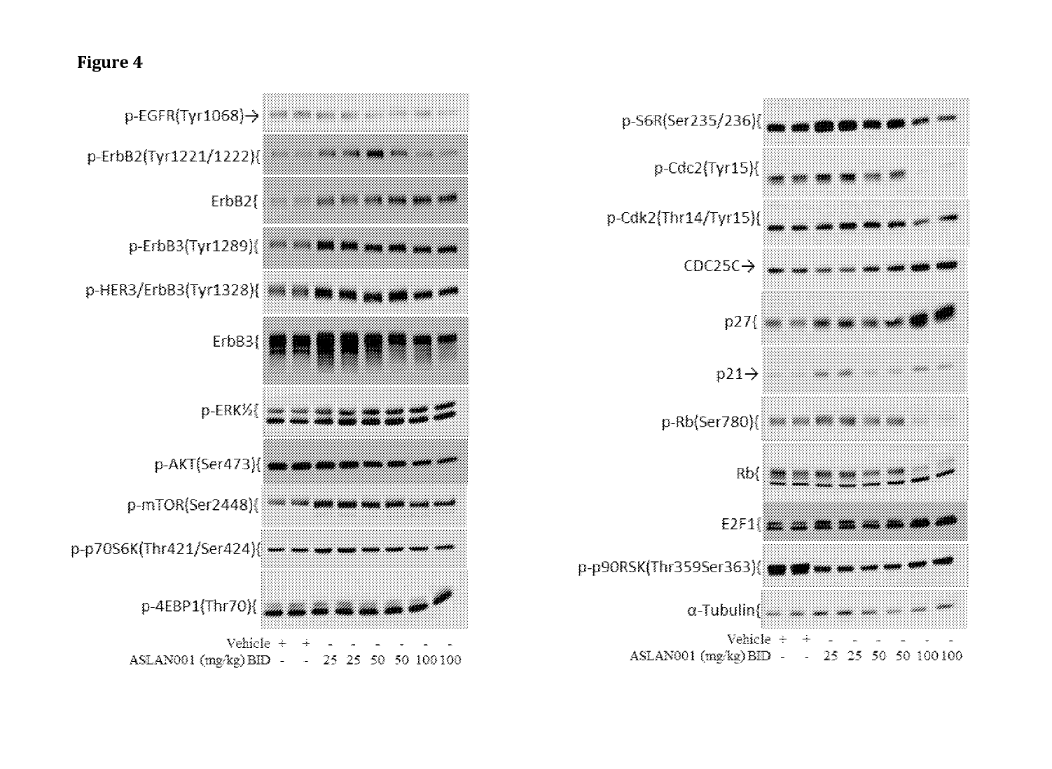

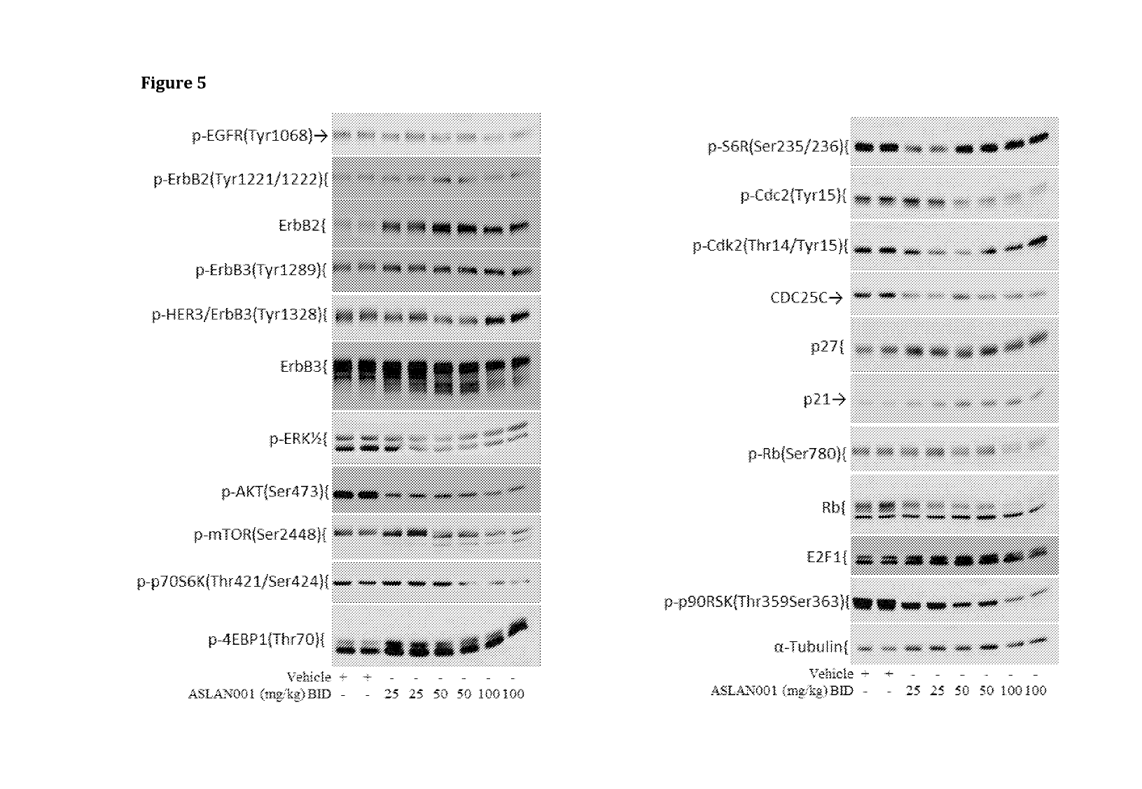

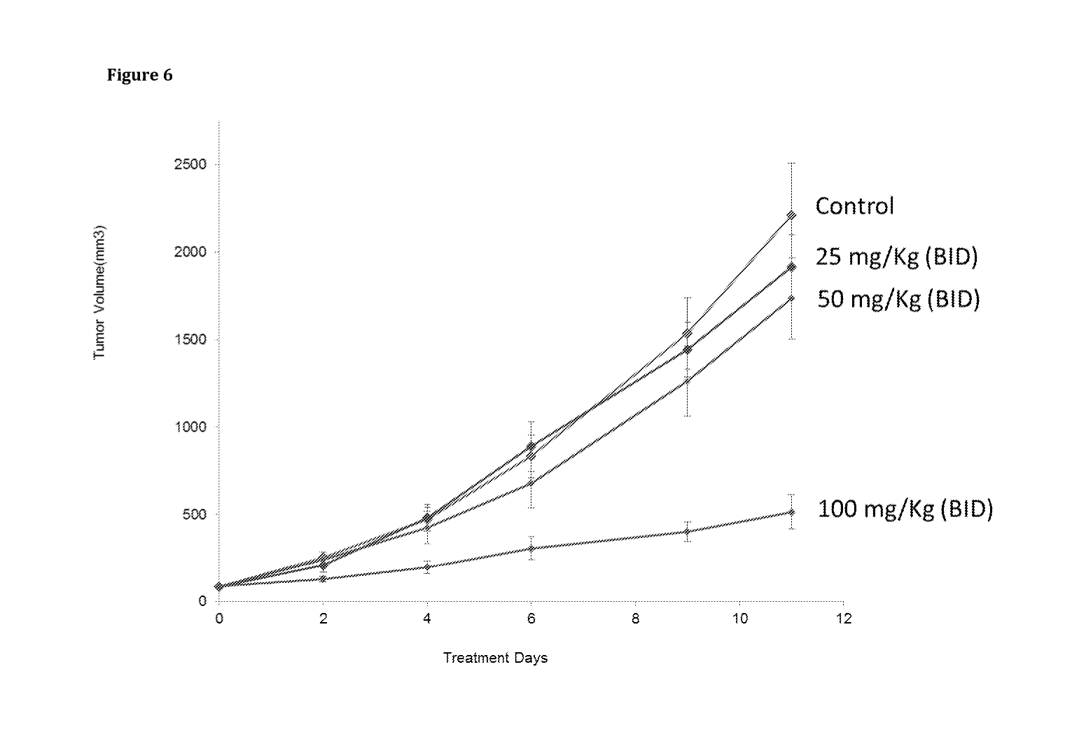

FIGS. 4-6 shows data from another HER2/HER3 co-expressing PDX model of HCC, HCC01-0708 after administration of 25 mg/kg 50 mg/kg and 100 mg/kg of varlitinib. The data generated showed that varlitinib inhibited tumour growth in the HCC01-0708 PDX model. The data also showed robust inhibition of the MAPK pathway after twelve days of treatment.

### Example 3

An in vitro experiment was conducted to investigate whether varlitinib induced apoptosis in HCC cell lines.

HCC cells were grown in cell culture medium with 10% FBS and with varying concentrations of varlitinib. Apoptosis profiles were analysed at 24 hour and 48 hour timepoints, using Muse Annexin V & Dead Cell Assay kit and Muse Cell Analyser. Early apoptosis cells (identified as Annexin V-PE positive and Dead Cell Marker negative) were measured and plotted.

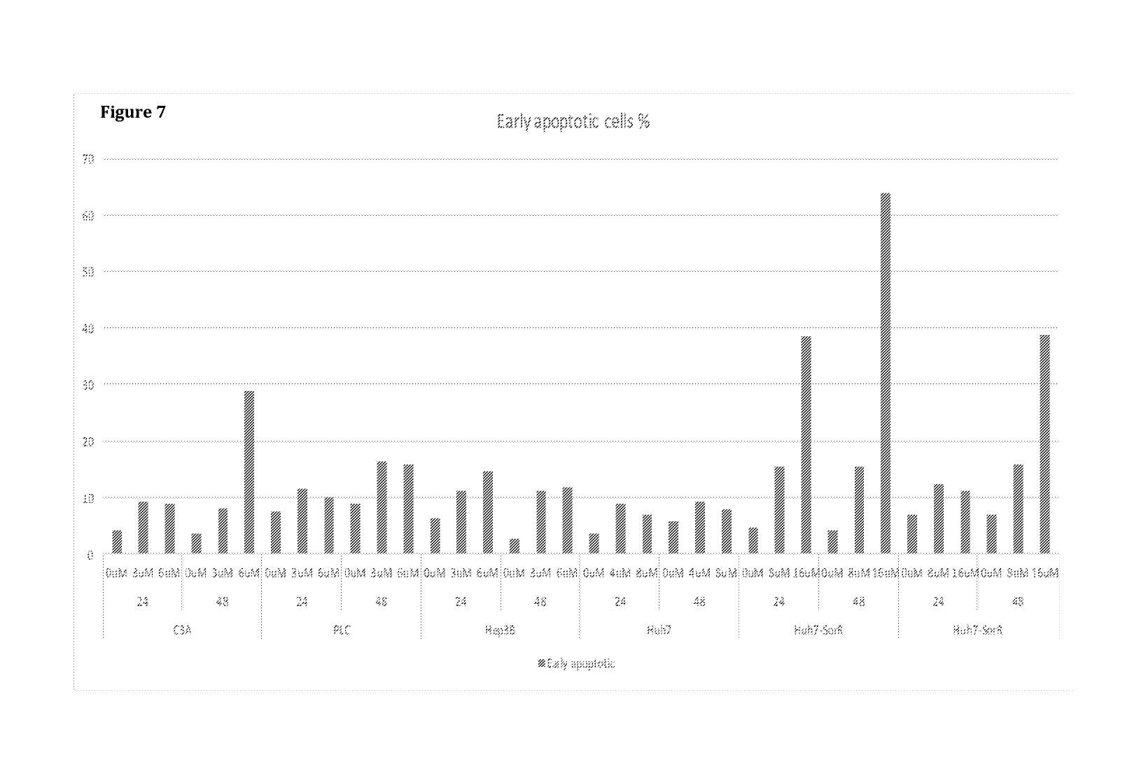

FIG. 7 shows the results of the experiment. As can be seen, varlitinib was able to induce early apoptosis in all of the HCC cell lines tested after 48 hours of incubation. Varlitinib was particularly effective in sorafenib resistant cells (Huh7-SorR) where almost 70% early apoptosis was observed when high dose of varlitinib was used, suggesting that varlitinib could be effective in patients who progress on sorafenib.

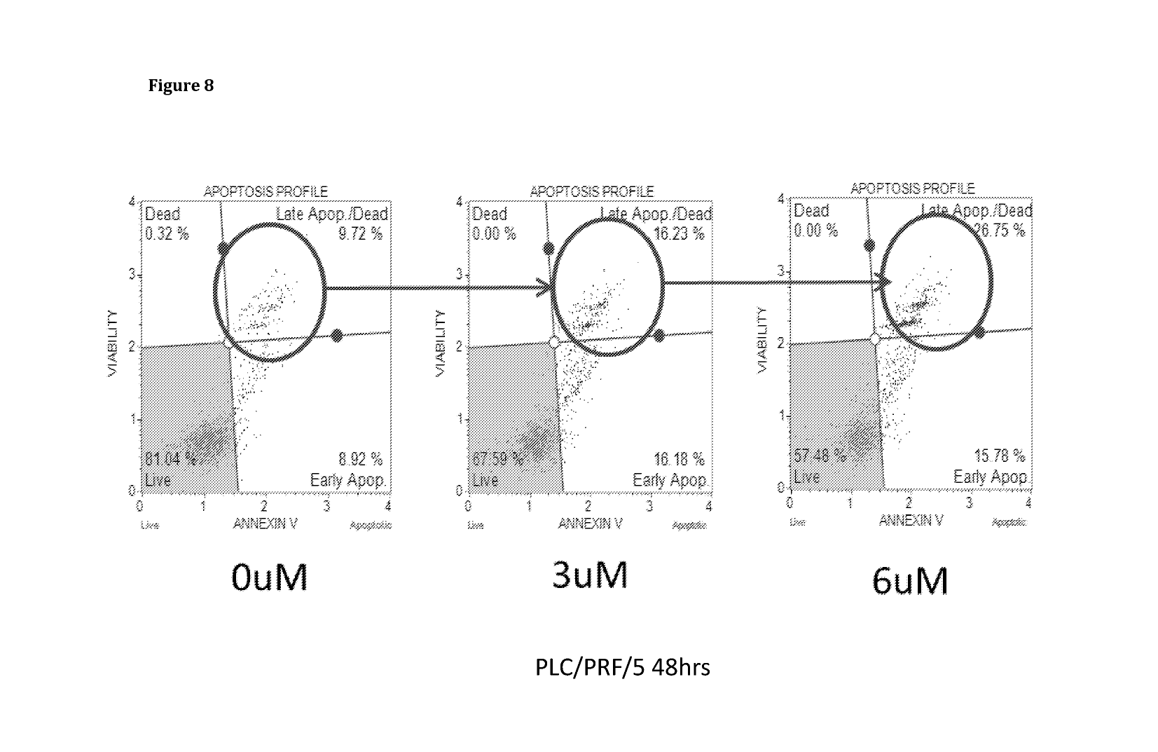

FIG. 8 shows the apoptosis profile for the PLC/PRF/5 (PLC) cells after 48 hour culture in the presence of varlitinib. Note the increase in percentage of apoptotic cells correlates with increasing varlitinib concentration.

Taken together, the in vitro results suggest the strong potential of varlitinib for use as a treatment for HCC, in particular refractory HCC.

The present inventors are also generating data where human patients are treated according to the present disclosure. The results are expected to be positive and will be available in due course

1. A method of treating a hepatocellular carcinoma patient by administering a therapeutically effective amount of a compound of formula (I): 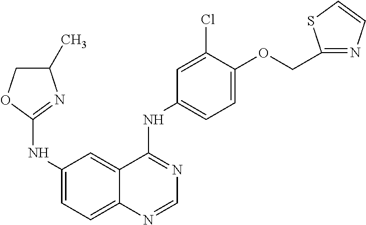 [SMILES: CC1COC(Nc2ccc3ncnc(Nc4ccc(OCc5nccs5)c(Cl)c4)c3c2)=N1]
an enantiomer thereof, or a pharmaceutically acceptable salt thereof.

2. A method according to claim 1, wherein the compound of formula (I) is: 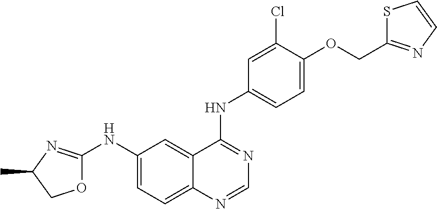 [SMILES: C[C@@H]1COC(Nc2ccc3ncnc(Nc4ccc(OCc5nccs5)c(Cl)c4)c3c2)=N1]
or a pharmaceutically acceptable salt thereof.

3. A method according to claim 1, wherein the compound of formula (I) is provided as the free base.

4. A method according to claim 1, wherein the compound of formula (I) is administered as a pharmaceutical formulation.

5. A method according to claim 1, wherein the compound of formula (I) or a pharmaceutical formulation comprising same is administered orally.

6. A method according to claim 1, wherein the compound of formula (I) or a pharmaceutical formulation comprising the same is administered bi-daily.

7. A method according to claim 1, wherein each dose of the compound of formula (I) is in the range 100 to 900 mg.

8. A method according to claim 7, wherein each dose of the compound of formula (I) is in the range 200 to 500 mg.

9. A method according to claim 8, wherein each dose is 400 mg.

10. A method according to claim 1, wherein the compound of formula (I) or pharmaceutical formulation comprising the same is employed as a monotherapy.

11. A method according to claim 1, wherein the compound of formula (I) or pharmaceutical formulation comprising the same is employed in a combination therapy.

12. A method according to claim 11, wherein the combination therapy comprises a chemotherapeutic agent selected from the group comprising doxorubicin, a platin (such as cisplatin or oxaliplatin), gemcitabine, capecitabine, 5-FU, FOLFOX, FOLFIRI and FOLFIRINOX.

13. A method according to claim 11, wherein the combination therapy comprises a targeted therapy selected from the group comprising sorafenib or a FGFR inhibitor.

14. A method according to claim 11, wherein the combination therapy comprises PD-1 or a PDL-1, or any other immune checkpoint inhibitor

15. A method according to claim 11, wherein the combination therapy comprises a DHODH inhibitor, for example 2-(3, 5-difluoro-3′-methoxybiphenyl-4-ylamino) nicotinic acid or a pharmaceutically acceptable salt thereof.

16. (canceled)

17. A method according to any one of claim 1, wherein the patient has refractory cancer.

18. A compound of formula (I): 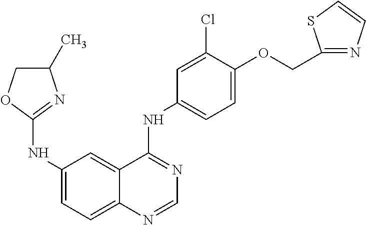 [SMILES: CC1COC(Nc2ccc3ncnc(Nc4ccc(OCc5nccs5)c(Cl)c4)c3c2)=N1]
an enantiomer thereof, or a pharmaceutically acceptable salt thereof for use in the treatment of hepatocellular carcinoma.

19. Use of a compound of formula (I): 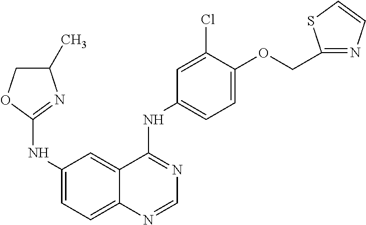 [SMILES: CC1COC(Nc2ccc3ncnc(Nc4ccc(OCc5nccs5)c(Cl)c4)c3c2)=N1]
an enantiomer thereof, or a pharmaceutically acceptable salt thereof in the manufacture of a medicament for the treatment of hepatocellular carcinoma.
